(19) United States
(12) Patent Application Publication
(10) Pub. No.: US 2005/0250110 A1
(43) Pub. Date: Nov. 10, 2005

(54) PNA conjugate for treating chronic myeloid leukemia (CML)

(76) Inventors: Klaus Braun, Sandhausen (DE); Waldemar Waldeck, Laudenbach (DE); Rudiger Pipkorn, Heidelberg (DE); Isabell Braun, Colbe-Burgeln (DE); Jurgen Debus, Heidelberg (DE)

Correspondence Address: Schwegman, Lundberg, Woessner & Kluth, P.A., P.O. Box 2938, Minneapolis, MN, 55402, US

(21) Appl. No.: 10/839,966

(22) Filed: May 6, 2004

Related U.S. Application Data
(63) Continuation of application No. PCT/DE02/04154, filed on Nov. 8, 2002.

(30) Foreign Application Priority Data
Aug. 11, 2001 (DE) 101 54 827.3

(51) Int. Cl. C12Q 1/68; C12P 21/04; C07K 14/47

(52) U.S. Cl. 435/6; 530/352; 435/69.7; 435/320.1; 435/325

(57) ABSTRACT

The invention relates to peptide nucleic acid (PNA) conjugates which inhibit the expression of the bcr/abl fusion gene characteristic of CML and are suited to treat chronic myeloid leukemia (CML).

### RELATED APPLICATIONS

This application is a continuation under 35 U.S.C. 111(a) of PCT/DE02/04154, filed on Nov. 8, 2002 and published on May 15, 2003 as WO 03/039438 A2, which claimed priority under 35 U.S.C. 119 of German Application No.: 101 54 827.3, filed Nov. 8, 2001, which applications and publications are incorporated herein by reference.

The present invention relates to peptide nucleic acid (PNA) conjugates which inhibit the expression of the bcr/abl fusion gene characteristic of CML and are suited for treating chronic myeloid leukemia (CML).

Chronic myeloid leukemia (CML) is the most frequent myeloproliferative disorder. A characteristic chromosome aberration was shown in connection therewith for the first time in the case of a hematological disease. The pathopysiologically relevant bcr/abl fusion gene usually forms as a result of a translocation t(9;22) and can be found on the shortened chromosome 22, i.e. what is called the Philadelphia chromosome (Ph). The corresponding gene product is a protein (p210) having tyrosine kinase activity. Over 95% of the CML patients are BCR/ABL-positive. The formation of the fusion gene in hematopoietic stem cells is the essential step for changing the regulatory behavior of the stem cells and results in a characteristic hematological and clinical picture of CML. In the case of a suppressed BCR-ABL protein, these abnormities are strongly reduced. This resulted in different therapy approaches which substantially aim at a specific elimination of the BCR-ABL expression.

As to the current therapy approaches it should be noted that the allogenic stem cell transplantation represents the sole curative therapy approach for the time being. However, this therapy is highly toxic and limited to relatively young CML patients having appropriate donors. Conventional strategies for treating CML are based on a therapy using interferon-α and hydroxy urea—inter alia in combination with cytarabine. Experimental methods, such as autologous stem cell transplantation, are also discussed for the time being. Reports are also found on a favorable course of clinical studies which are based on a cABL-specific inhibitor (STI 571). Yet STI 571 seems to also interact with further tyrosine kinases other than the BCR-ABL protein for lack of specificity. In this connection, it should also be noted that the primitive CML progenitor cells are likely not to express either BCR-ABL mRNA or p210 and are thus not available to a therapy using ST 571 and the classical antisense therapy directed against mRNA. As to the formerly conducted antisense studies, it should be noted that they are unsatisfactory since they are ineffective for lack of sufficient biological half life of the employed antisense oligonucleotides as compared to the half life of the BCT-ABL mRNA. The problem of an efficient transfer of the antisense oligonucleotides to the destination by biological membranes has not yet been solved either. Moreover, all of the patients treated with the above-mentioned, previously established therapies suffered from leukemic relapses accompanied by an unfavorable prognosis. Since these therapies were thus unable to achieve a decisive break-through in the CML treatment, the prognosis of success is currently unfavorable and clearly shows the necessity of more effective therapies.

Thus, the invention is substantially based on the technical problem of providing products which permit an effective therapy of chronic myeloid leukemia (CML).

This technical problem is solved by the provision of the embodiments characterized in the claims. In order to achieve the solution to this technical problem, the inventors developed a conjugate and a conjugate mixture comprising the following components: (a) a transport mediator for the cell membrane (P), (b) and address protein or peptide (AP) for an import into the nucleus, and (c) a peptide nucleic acid (PNAs) to be transported and specifically hybridizable with the fusion gene bcr/abl, which inhibits the expression thereof.

These modular conjugates have two decisive advantages:

(a) Components P and AP enable an efficient and directed transport of PNA to the destination and thus a gene therapy. These components do not only permit a rapid and effective transport of PNA through the cell membranes of living cells into the cytoplasm but, following the cytoplasmic activation of address peptide sequences, also an efficient transport into the nucleus.

(b) The use of protease- and nuclease-resistant peptide nucleic acids (“PNAs”) which are oligonucleotide derivatives where the sugar phosphate backbone is preferably substituted by ethyl-amine-bound α-amino-ethyl-glycine units, permits the stable and efficient blocking of the transcription of the desired genes under physiological conditions on account of their physico-chemical properties. By means of these PNAs an anti-gene strategy based on the anti-sense principle is pursued where it is not the mRNA but the gene per se that is the target. Here, the PNAs hybridize via the formation of a triple helix to the target DNA and can thus inhibit the synthesis of the fusion protein p210. On account of the specificity of the PNA conjugates according to the invention it is possible to distinguish individually recombinant fusion bcr/abl DNA from unchanged, non-fused bcr and abl DNA.

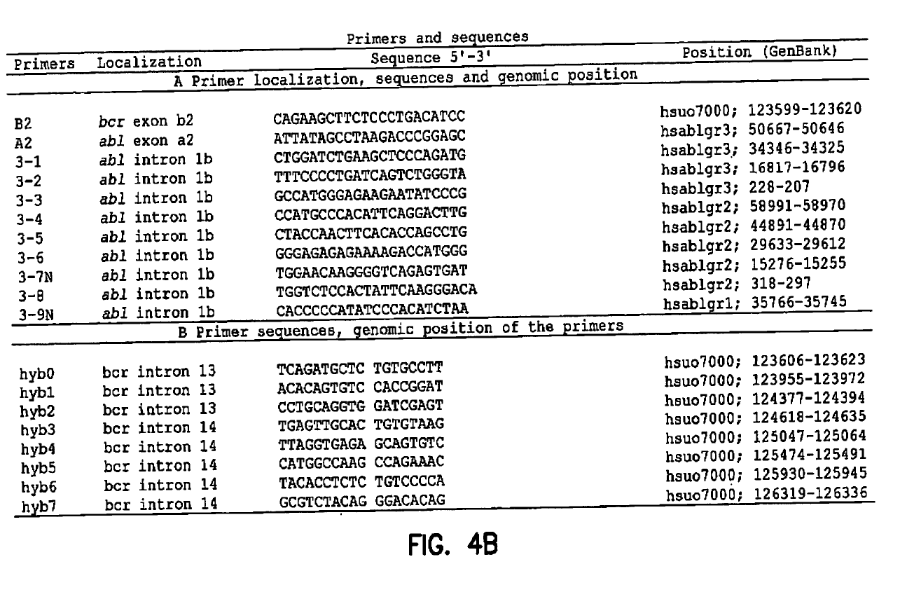

In summary, it can be found that the conjugates according to the invention represent a new kind of drugs which are transported effectively to the destination and on account of their high stability and excellent specificity enable a stable and efficient transcription control of bcr/abl at a genomic level. The PNA conjugates according to the invention should be effective even with extremely low administration doses (below 100 pM final concentration) so as to prevent, or at least strongly reduce, the occurrence of undesired side-effects. The specificity of the PNA conjugates according to the invention is achieved by characterizing recombination hotspots using established methods, such as PCR, subsequent sequencing and identification by means of biocomputing methods, and on this basis the PNA sequences of the conjugates according to the invention are designed for an individual active substance design. It was possible to prove this by means of the proliferation stop of the tumor cell line K562 (Ph-positive) (Grosveld et al., Molecular and Cellular Biology 6 (2) (1986), 607-616) by way of experiment in the below Example 3 (see also FIG. 5).

Thus, the present invention relates to a conjugate for the specific inhibition of the expression of a bcr/abl fusion gene, the conjugate comprising the following components: (a) a transport mediator for the cell membrane, (b) an address protein or peptide for the import into the cell compartments, preferably the nucleus, and (c) a peptide nucleic acid (PNA) specifically hybridizable with the fusion gene bcr/abl, which inhibits the expression thereof.

As to methods of producing the individual components of the conjugates and the linkage thereof reference is made to German patent application No. 199 33 492.7. The synthesis of PNAs is known to the person skilled in the art and also described in Nielsen et al., Science 254 (1991), 1497-1500, for example.

The structure of the conjugate according to the invention is preferably as follows: P-AP—PNA_{bcr/abl-specific}, more preferably it is P—S—S-AP-spacer-PNA_{bcr/abl specific}, —S—S— corresponding to a disulfide bridge.

The transport mediator for the cell membrane is preferably a peptide or protein which can penetrate the plasma membrane. The length of this peptide or protein is not limited as long as it has the above property. Transport mediators are of viral, bacterial or human origin, for example, and are derived preferably from the penetratin family (Derossi et al., Trends Cell Biol. 8 (1988), pages 84-87), are transportan or parts thereof (Pooga et al., The Faseb Journal 12 (1998), page 68 et seq.), the transmembrane peptide pAntp(43-58), TPU^{Eco}, preferred amino acid sequence: H₂N-MTRQTFWHRIKH—COOH, TPU^{HIV-1-TAT}, preferred amino acid sequence: H₂N—YGRKKRRQRRR—COOH, or TPU^{Hum}, preferred amino acid sequence: H₂N—KMTRQTWWHRIKHKC-(Cys-CO—NH₂)—(SH)—CONH₂, H₂N-MTRQTFWHRIKHKC-(Cys-CO—NH₂)—(SH)—CONH₂ or H₂N—KHKIRHWFTQRTMC-(Cys-CO—NH₂)—(SH)—CONH₂.

The selected transport mediator is produced biologically (purification of natural transport mediator proteins or cloning and expression of the sequence in a eukaryotic or prokaryotic expression system), and preferably synthetically, e.g. according to the Merrifield method (Merrifield, J. Am. Chem. Soc. 85 (1963), 2149).

A person skilled in the art can select the address protein or peptide by means of the known amino acid sequences for the import into peptides or polypeptides controlling the nucleus. In principle, the length of this address peptide or protein is not limited as long as it has the property of ensuring a nucleus-specific transport. In order to introduce the PNAs, address proteins or address peptides are generally selected which contain a nucleus-specific recognition signal and thus send the PNAs into the nucleus. Fundamentally, the pure address sequence suffices for the transport into the nucleus. However, it is also possible to select address proteins/address peptides which have a cytoplasmic peptidase cleavage site. In the most favorable case, this cleavage site is within the signal sequence but can also be attached thereto by additional amino acids to ensure the cleavage of the address sequence after reaching the cytoplasm. The selected “AP” sequence is produced biologically (purification of natural transport mediator proteins or cloning and expression of the sequence in a eukaryotic or prokaryotic expression system), and preferably synthetically, e.g. according to the Merrifield method (Merrifield, J. Am. Chem. Soc. 85 (1963), 2149). Examples of suitable address proteins or peptides are as follows: (a) -Pro-Pro-Lys-Lys-Lys-Arg-Lys-Val and (b) H₃N⁺-Pro-Lys-Lys-Lys-Arg-Lys-Val- (=nuclear localization sequence (NLS) from the SV40 T antigen).

The conjugate can also optionally contain a spacer (above abbreviated as SP) which is preferably located between the address protein/peptide and the peptide nucleic acid (PNA) to be transported. However, in addition or alternatively it can also be present between the transport mediator and the address protein. The spacer serves for eliminating or favorably influencing optionally existing steric interactions between the components. For example, the spacer can be selected from polylysine, polyethylene glycol (PEG), derivatives of poly-methacrylic acid or polyvinyl pyrrolidone (PVP).

A redox cleavage site, e.g. -cysteine-S—S-cysteine-O—N—H—, is preferably located between the transport mediator and the address protein/peptide. The bond resulting between transport mediator and address protein is a redox coupling (mild cell-immanent linkage by means of DMSO; Rietsch and Beckwith, Annu. Rev. Gent 32 (1998), 163-84):
Cysteine-SH SH-cysteine---→cystine-S—S-cystine

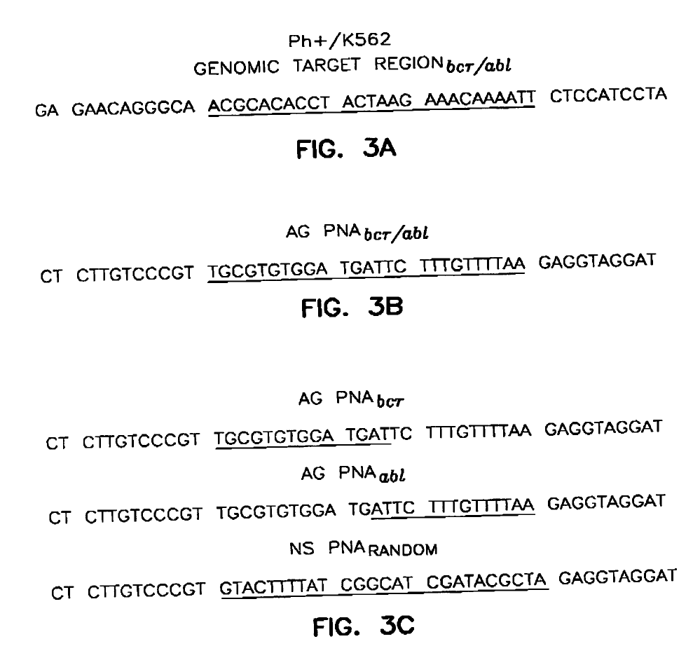

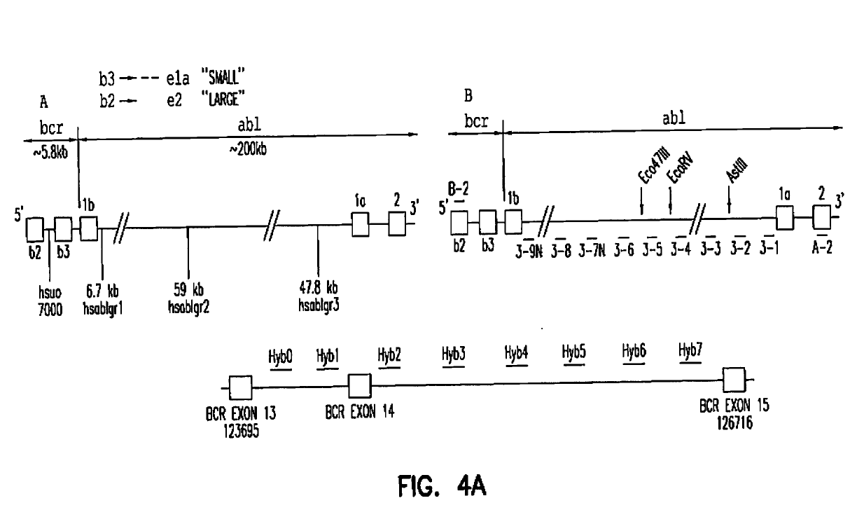

The peptide nucleic acid (PNA) of the conjugates according to the invention permits the specific inhibition of the transcription of the bcr/abl fusion genes thereby avoiding the inhibition of the native, non-recombinant genes bcr and/or abl by hybridizing them with a fusion gene region which is transcribed and contains the recombination point. Suitable regions may be determined by the person skilled in the art, e.g. by means of the method described in below Example 2, which may finally result in an establishment of a DNA library of Ph-chromosome-positive samples and a subsequent identification of individual recombination loci. Individual conjugates matched specifically to the patients can also be produced by removing CML cells therefrom and culturing them, then determining the recombination point e.g. by means of the molecular site of action shown in FIG. 3 on the genomic DNA and by means of the primers listed in FIG. 4 and the LT-PCR described in Example 2 and sequencing, and designing the corresponding matching PNA conjugates, i.e. PNA conjugates having sequences hybridizing specifically to the bcr/abl recombination point only. For the LT-PCR primers, it would be advantageous to select primers having a length within the region of 25 bp, an attachment temperature of 68 to 69° C., a GC primer content of about 55% and a primer concentration of about 20 pmol per PCR batch. The peptide nucleic acids preferably have a length of at least 20 bases, peptide nucleic acids having a length of at least 23 bases being particularly preferred. The peptide nucleic acid can optionally be labeled, e.g. radioactively, using a dye, biotin/avidine, etc.

The conjugate constituents P and AP are synthesized preferably synthetically according to the Merrifield method (Merrifield, J. Am. Chem. Soc. 85 (1963), 2149). The other constituents (e.g. spacer and/or PNA) are attached thereto by covalent chemical bond. The redox cleavage site is inserted between P and AP chemically by the above-mentioned redox linkage. A covalent bond, preferably an acid amide bond, also exists between an optionally present spacer and the PNA or between the address protein and the PNA. Possible alternatives are ether or ester bonds, depending on the functional group(s) present in the substance to be conjugated.

In a particularly preferred embodiment of the conjugate according to the invention, the peptide nucleic acid (PNA) comprises the following sequence:

TGC GTG TGG ATG ATT CTT TGT TTT AA (orientation:
N-terminus/sequence/C-terminus).

Finally, the present invention also relates to a medicament containing a conjugate according to the invention, optionally together with a suitable carrier, and its use for treating CML. An intravenous administration is a preferred administration route.

The invention is further explained by means of the following figures:

Legend for the figures:

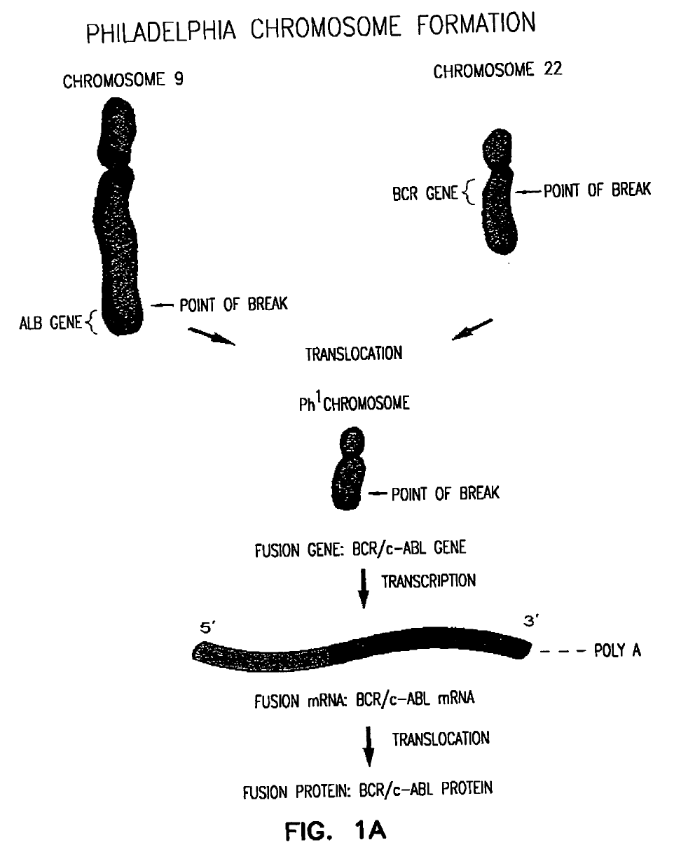

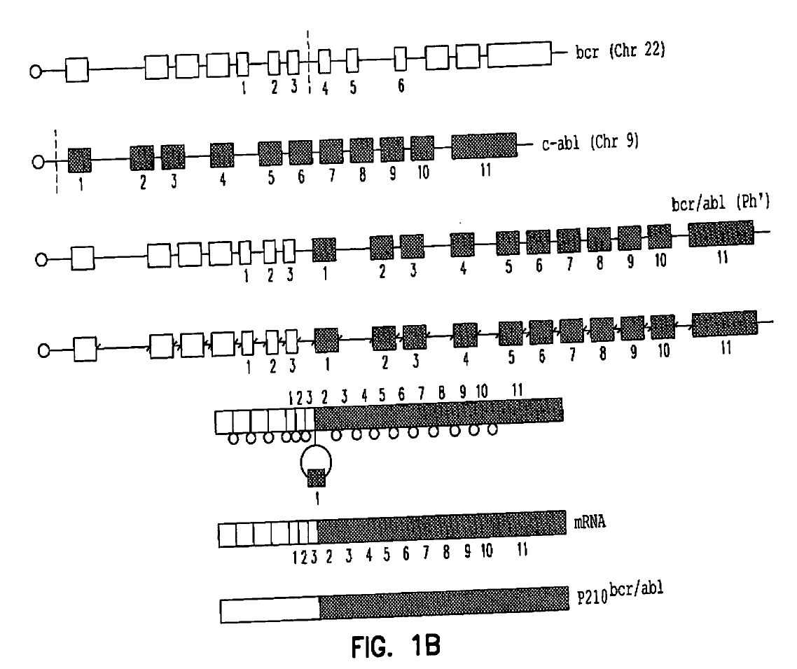

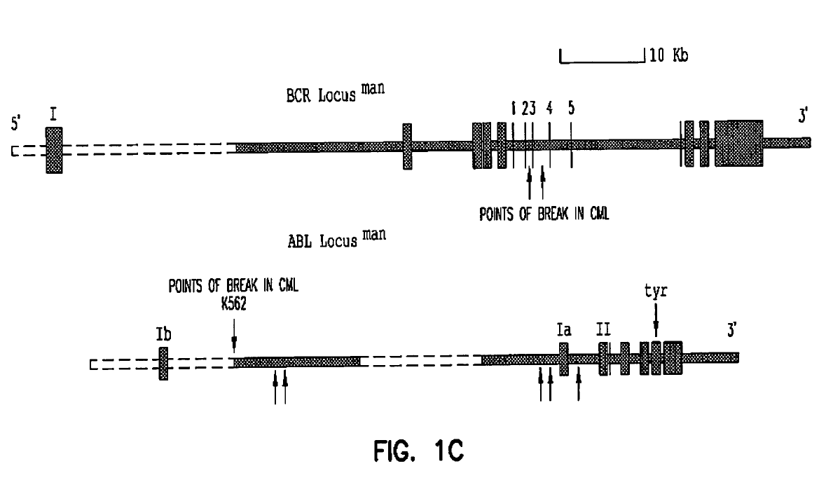

FIG. 1: Diagram of the formation of the Philadelphia chromosome by translocation between chromosomes 9 and 22

(A) overview

(B) diagram of the exon/intron structure of the bcr/abl fusion gene

(C) more detailed description of the points of break within the bcr gene and the abl gene

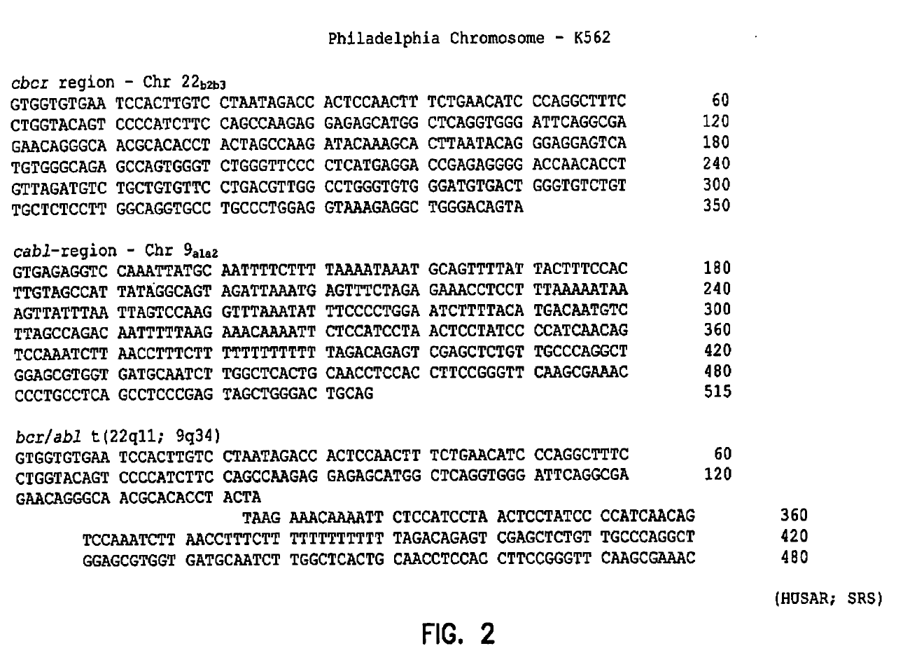

FIG. 2: Description of the genomic site of action within the region of the recombination locus of the “Philadelphia chromosome” using the cell line K562 as an example

Top region: DNA sequences of the original bcr gene on chromosome 22

Middle region: DNA sequences of the original abl gene on chromosome 9

Bottom region: DNA sequences around the recombination site between bcr gene and abl gene

FIG. 3: Description of the DNA sequences of the genomic target region of the K562-DNA for a PNA conjugate strategy

What is shown is

(A) the target region of the genomic bcr/abl DNA of the cell line K562,

(B) the corresponding sequence, used for a transcription inhibition, of a PNA conjugate, and

(C) different sequences for control PNA conjugates.

FIG. 4: LT-DNA-PCR primer design and localization

(A) diagram of the localization of the different primers within the bcr/abl fusion gene,

(B) description of the sequences of the different primers and their position (the “reverse” primers are indicated.).

FIG. 5: Growth behavior of K562 cells following the administration of different conjugates

The cell number change is shown on a time axis. The treatment was carried out throughout the experiment. Final concentration: 100 nM. Dark blue line: AS conjugate; magenta: random sequence conjugate; yellow: AS-non-ligated; blue: untreated control.

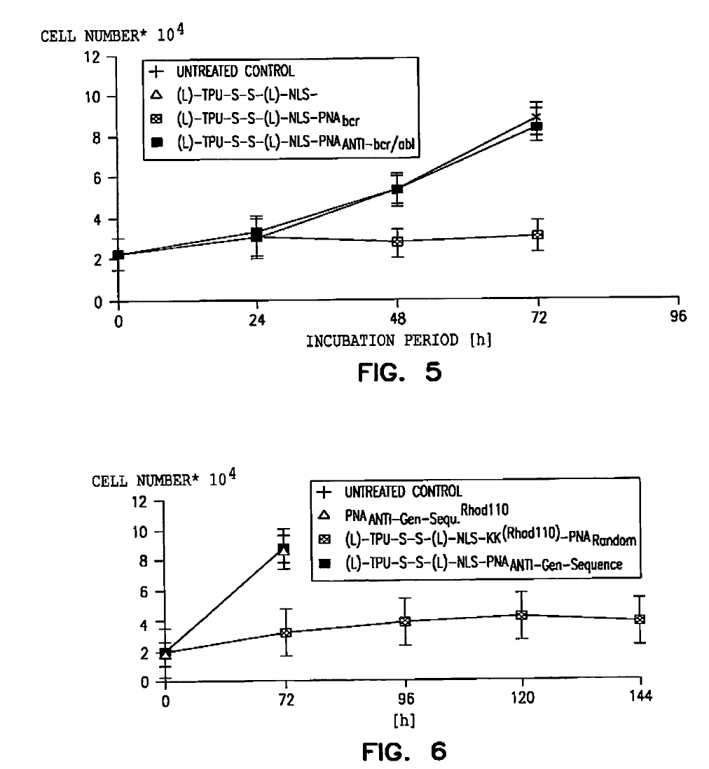

FIG. 6: Apoptosis behavior of K562 cells following the administration of different conjugates

The cell number change is shown on a time axis. The treatment was carried out for 24 h and by means of subsequent PNA removal. Final concentration: 100 nM. Dark blue line: AS conjugate; magenta: random sequence conjugate; yellow: AS-non-ligated; blue: untreated control.

The invention is further described by means of the below examples.

### EXAMPLE 1

### General Methods

(A) Peptide Synthesis

Peptide nucleic acid (PNA) imitates a DNA and was originally developed as a reagent for the sequence-specific recognition of double-stranded DNA via conventional triple helix formation. For the solid-phase synthesis, the Fmoc strategy was used by means of a fully automated synthesis device (Syro II, Multisyntech company, Witten, Germany). The synthesis was carried out with a 0.05 mmol Fmoc-AS polystyrene resin (1% cross-linked). 2-(1H-benzotriazol-1-yl)-1,1,3,3-tetramethyluroniumhexafluorophosphate (HBTU) was used as the coupling reagent. The groups protecting the side chains were Lys(Boc), Asp(Obut), Ser(But), Cys(Trt) and Asn(Trt). The protected peptidyl resin was treated with 20% piperidine in dimethylformamide. The cleavage and separation of the protecting groups were obtained by treatment with 90% trifluoroacetic acid, 5% ethanedithiol, 2.5% thioanisol and 2.5% phenol (v/v/v) at room temperature for 2.5 h. All of the products were precipitated in ether and purified by preparative HPLC (Shimazu LC-8A, Shimazu, Duisburg, Germany) on a YMC ODS-A 7A S-7 μm reverse phase HPLC column (20×250 mm) using 0.1% trifluoroacetic acid in water (A) and 60% acetonitrile in water (B) as an eluent. The peptides were eluted with a linear gradient of 25% B to 60% B within 40 min. at a flow rate of 10 ml/min. The fractions corresponding to the purified conjugate were lyophilized. Sequences of individual molecules and the complete bimodular construct were characterized by analytical HPLC (Shimadzu LC-10) and laser desorption mass spectroscopy (Vision 2000, Finnigan MAT, San Jose, Calif., U.S.A.) as pointed out below. The random PNA construct was produced with an identical carrier conjugate.

Peptide Purification:

Gradient:
analyt. 5%→80% (within a period of 35 min.);
preparative: 5%→80% (within a period of 40 min.);

Purity:
>90%.

The peptide was linked to the other conjugate constituents according to common methods (see the above description).

(B) Measurement of Cell Growth

The cell growth was measured by means of a “Light Cycler” device (Roche, Basel, Switzerland). The cell number was measured using a “Coulter Counter” device.

(C) Flow Cytometry

The effect of the PNA conjugates on the viability of the K-562 cells and the cell cycle distribution were determined by flow cytometry. The flow cytometry analyses were carried out with a “PAS II” flow cytometer (Partec, Münster, Germany) which was equipped with a 100 Watt mercury arc lamp and filter combinations for cells stained with 2,5-diamidino-2-phenylindole (DAPI). The cells were isolated from native samples at room temperature with mild shaking using 2.1% citric acid/0.5% Tween® according to the method by de Villiers et al., Gynecol. Oncol. 44 (1992), 33-39, with slight modifications. DAPI-containing phosphate buffer (7.2 g Na₂HPO₄×2H₂O in 100 ml H₂O), pH 8.0) was used for staining the cell suspensions. Every histogram represents 30,000 to 100,000 cells for measuring the DNA index and the cell cycle. In order to analyze the histograms, the “Multicycle” program by Phoenix Flow Systems, San Diego, Calif., was used. The viability of the cells was determined by a new flow cytometry method. To this end, the cells were incubated with the PNA conjugates (100 pM) for 24 or 48 hours. Untreated cells served as a control. The analyses were carried out in a “FACS Calibur” flow cytometer (Becton Dickinson Cytometrie Systems, San Jose, Calif.) using the forward and sideward scatter and the relative fluorescence intensities of the native cells stained using propidium iodide were measured on the F2 channel with logarithmic graduation. Dead cells are positive with respect to propidium diiodide and stained in red, living cells remain unstained.

### EXAMPLE 2

### Identification of Individual bcr/abl Recombination Loci Via LT-DNA PCR and Sequencing

Although the plurality of CML progenitor cells have the DNA recombination locus, not all of them express the bcr/abl hybrid mRNA or the gene product p210. These cells were not detected by the former diagnostic methods. This problem can be circumvented by the below described strategy by characterizing the recombination site at the DNA level. The chromosomal point of break is within the region of above 200 kbp and therefore calls for an LT-DNA PCR (long terminal DNA polymerase chain reaction). The below described specific primers enable a characterization of individual recombination loci irrespective of the mRNA expression and p210 translation.

(A) Sampling

Peripheral blood of 970 patients and 10 healthy control persons was used. Further controls were the Ph⁺ cell line K-562. Here, a differentiation was made into: CT-treated, INF-treated, STI-571-treated, untreated, stage.

(B) DNA Isolation

DNA was isolated from blood according to the manufacturer's instructions with the “Qiagen Blood and Cell Culture DNA Midi Kit” (Qiagen GmbH, Hilden, Germany). Here, about 100 μg genomic DNA was obtained from about 10⁷ cells each. This DNA was resuspended in Tris-EDTA buffer and frozen at −20° C.

(C) LT-DNA PCR Primer Design

BCR and Abl exons were used as a basis and then screened step-wise after about 40 kbp in the genomic database “HUSAR” for appropriate “reverse” primers. A primer pair which hybridizes in exon b2 of bcr and exon a2 of abl was used for the introductory PCR and primers which hybridize in exon 15 of bcr and exon 2 of abl were used for the actual LT-DNA PCR (see FIG. 4B). The abl localization is based on the GenBank sequence (AC: UO7561), “ABL-PK” referring to constant primer in ABL-Exon 2. The bcr localization is based on the GenBank sequence (UO7000), “BCR-PK” referring to constant primer in BCR-Exon 15. Here, PCR products are obtained in the region of about 50 kbp. Regarding the LT-PCR, a kit was used which contains the “Advantage© Genomic” polymerase (Clontech Laboratories, Palo Alto, Calif., U.S.A.) by means of which PCR products up to about 40 kpb can be obtained, which permits major steps regarding the search in genomic databases and thus represents a model basis for “Genomic Walking”. The LT-PCR reaction batch contained: about 20 ng genomic DNA, 2 μl dNTP-Mix (10 mM), 0.1 U/μl polymerase, 1.5 mM MgCl₂, 20 pmol primer (according to FIG. 4B), total number of cycles: 25-30. The amplified products were applied onto a 1.4% agarose gel and the DNA was isolated from the corresponding bands by common methods and sequenced.

### EXAMPLE 3

### Inhibition of the Proliferation of the Tumor Cell Line K562 Following the Administration of the PNA Conjugates According to the Invention

A conjugate having the following structure was used for these studies: Transport peptide (penetratin)-disulfide bridge-NLS—KK_{(FITC)}—PNA^{BCR/Abl} (sequence of PNA^{BCR/Abl}: H₂N-TGC GTG TGG ATG ATT CTT TGT TTT AA-COOH). The PNA sequence of the random conjugate is evident from FIG. 3.

(A) Experimental Procedure

The conjugate was purified by means of analytical HPLC. The conjugate was prepared as a parent solution in 0.9% NaCl solution (sterile). The K562 cells (fresh suspension) were cultured in 10 ml Falcon culture bottles (flat bottom) in RPMI 1649 medium (Gibco) (37° C., 5% CO₂) for 24 h. The cells were treated with the conjugate according to the following protocol prior to the removal of the RPMI medium:

Batch 1 (proliferation) (“Coulter Counter” method): 50 μl conjugate parent solution, 4950 μl cell suspension (K562, about 1000 cells per ml).

Batch 2 (CMLS): 102.4 μg (substance)=4.5 ml total volume (RPMI medium); volume glass carrier with 8 chambers (300 ml per chamber).

For this purpose, Falcon bottles having 5 ml prepared cell suspension each were treated for the time measuring points as follows: The RPMI medium was removed, replaced by the same volume of the mixture according to batch 2 and incubated at 37° C. in a 5% CO₂ atmosphere for 1 hour. The gene expression was then controlled according to the temperature at 37° C. (control). The localization and the reaching of the destination were determined at the same time by means of CLSM. For this purpose, the cells were adhered to 8-chamber slides using ZellTak® over one hour before the measurement. The medium was removed and washed twice with dye-free RPMI medium. Following the embedding step using Mowiol®, the FITC fluorescence was determined directly via CLSM. Analogous: excitation 488 nm, emission: 520 nm. The co-localization of the NLS partial conjugate was determined by the addition of Lysotrek®. The nucleus staining (DAPI) was carried out by Vectashield® treatment (Alexis Biochemicals, Grünberg, Germany) instead of a Mowiol® treatment and determined by means of a U.V. laser.

(B) Growth Behavior of K562 Cells (See FIG. 5)

The samples and controls were treated identically. The final active substance concentrations in the RPMI 1640 medium (colorless) were 100 nM. The incubation periods corresponded to the duration of the experiment. It turned out that neither the conjugate random sequence PNA (magenta) nor the anti-gene PNA without transporter (yellow) showed a difference to the untreated control as regards the growth behavior. After 72 hours, the cell numbers of the control cultures were no longer determinable on account of a chaotic growth behavior. The K562 tumor cells treated with antisense NLS penetratin (dark blue) showed a stagnation of the cell proliferation (stabilization of the cell number 24 hours after the treatment). The cells also showed a vital phenotype in contrast to the controls. Similar results were obtained with conjugates which differed as regards the transport mediator (bacterial (TPU^{Eco}), preferred amino acid sequence: H₂N-MTRQTFWHRIKH—COOH), (viral (TPU^{HIV-1/TAT)}, preferred amino acid sequence: H₂N—YGRKKRRQRRR—COOH, human (TPU^{Hum})).

### EXAMPLE 4

### Apoptosis Behavior of the Tumor Cell Line K562 Following the Administration of the PNA Conjugates According to the Invention

The question of the apoptosis behavior following the administration of a conjugate according to the invention (corresponding to Example 3 and FIG. 6) was studied as such. The incubation period was 24 hours with 5% CO₂ at 37° C. in an incubator. Having removed the medium mixture and carrying out a wash step twice using fresh RPMI-1640, the same volume of fresh medium was added by pipetting and further cultured. The medium was changed every 72 hours. The cultures were treated as before: As in the previous experiment, a break-down of the untreated culture was again found after about 72 hours. Uncoupled antisense PNA and random sequence NLS-PNA conjugates showed no difference as regards the cell growth. The relative stability up to 144 hours following the removal of antisense NLS-PNA was striking in the culture treated with antisense NLS-PNA. Furthermore, a minor increase in the cell number up to a maximum at 120 hours was found following PNA removal.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A conjugate for specifically inhibiting expression of a bcr/abl fusion gene, wherein the conjugate comprises:
(a) a transport mediator for a cell membrane;
(b) an address protein or peptide for import into the nucleus; and
(c) a peptide nucleic acid (PNA) hybridizable specifically with the fusion gene bcr/abl and inhibiting the expression thereof.

2. The conjugate according to claim 1, wherein the transport mediator is a peptide or protein which can penetrate the plasma membrane.

3. The conjugate according to claim 1, wherein the transport mediator is transmembrane peptide pAntp(43-58), TPU^{Eco}, TPU^{HIV-1/TAT} or TPU^{Hum}.

4. The conjugate according to claim 1, wherein the address protein or peptide is selected from:
-Pro-Pro-Lys-Lys-Lys-Arg-Lys-Val-; and
H₃N⁺-Pro-Lys-Lys-Lys-Arg-Lys-Val-.

5. The conjugate according to claim 1, wherein a spacer is additionally present.

6. The conjugate according to claim 5, wherein the spacer is located between the address protein and peptide nucleic acid (PNA).

7. The conjugate according to claim 6, wherein the conjugate has the following structure: transport mediator-S—S-address peptide-spacer-PNA.

8. The conjugate according to claim 7, wherein the peptide nucleic acid comprises the sequence TGC GTG TGG ATG ATT CTT TGT TTT AA.

9. A pharmaceutical composition comprising a conjugate according to claim 1.

10. A method for treating chronic myeloid leukemia comprising administering the conjugate of claim 1.


SEQUENCE LISTING (35 sequences)

SEQ ID NO: 1 - protein, 12 aa, E. coli
MTRQTFWHRIKH

SEQ ID NO: 2 - protein, 11 aa, Human immunodeficiency virus type 1
YGRKKRRQRRR

SEQ ID NO: 3 - protein, 16 aa, Homo sapiens
KMTRQTWWHRIKHKCC

SEQ ID NO: 4 - protein, 15 aa, Homo sapiens
MTRQTFWHRIKHKCC

SEQ ID NO: 5 - protein, 15 aa, Homo sapiens
KHKIRHWFTQRTMCC

SEQ ID NO: 6 - protein, 8 aa, artificial sequence
  addressprotein
PPKKKRKV

SEQ ID NO: 7 - protein, 7 aa, Simian virus 40
PKKKRKV

SEQ ID NO: 8 - DNA, 26 bp, artificial sequence
  PNA-conjugate
tgcgtgtggatgattctttgttttaa

SEQ ID NO: 9 - DNA, 350 bp, K 562
gtggtgtgaatccacttgtcctaatagaccactccaactttctgaacatcccaggctttcctggtacagtccccatcttccagccaagaggagagcatggctcaggtgggattcaggcgagaacagggcaacgcacacctactagccaagatacaaagcacttaatacagggaggagtcatgtgggcagagccagtgggtctgggttcccctcatgaggaccgagaggggaccaacacctgttagatgtctgctgtgttcctgacgttggcctgggtgtgggatgtgactgggtgtctgttgctctccttggcaggtgcctgccctggaggtaaagaggctgggacagta

SEQ ID NO: 10 - DNA, 395 bp, K 562
gtgagaggtccaaattatgcaattttcttttaaaataaatgcagttttattactttccacttgtagccattataggcagtagattaaatgagtttctagagaaacctcctttaaaaataaagttatttaattagtccaaggtttaaatatttcccctggaatcttttacatgacaatgtcttagccagacaatttttaagaaacaaaattctccatcctaactcctatccccatcaacagtccaaatcttaacctttctttttttttttttagacagagtcgagctctgttgcccaggctggagcgtggtgatgcaatcttggctcactgcaacctccaccttccgggttcaagcgaaacccctgcctcagcctcccgagtagctgggactgcag

SEQ ID NO: 11 - DNA, 308 bp, K 562
gtggtgtgaatccacttgtcctaatagaccactccaactttctgaacatcccaggctttcctggtacagtccccatcttccagccaagaggagagcatggctcaggtgggattcaggcgagaacagggcaacgcacacctactataagaaacaaaattctccatcctaactcctatccccatcaacagtccaaatcttaacctttctttttttttttttagacagagtcgagctctgttgcccaggctggagcgtggtgatgcaatcttggctcactgcaacctccaccttccgggttcaagcgaaac

SEQ ID NO: 12 - DNA, 48 bp, K 562
gagaacagggcaacgcacacctactaagaaacaaaattctccatccta

SEQ ID NO: 13 - DNA, 48 bp, artificial sequence
  PNA-conjugate
ctcttgtcccgttgcgtgtggatgattctttgttttaagaggtaggat

SEQ ID NO: 14 - DNA, 48 bp, artificial sequence; identical to SEQ ID NO: 13
  control conjugate

SEQ ID NO: 15 - DNA, 48 bp, artificial sequence; identical to SEQ ID NO: 13
  control conjugate

SEQ ID NO: 16 - DNA, 48 bp, artificial sequence
  control conjugate
ctcttgtcccgtgtacttttatcggcatcgatacgctagaggtaggat

SEQ ID NO: 17 - DNA, 22 bp, control conjugate
  Primer B2
cagaagcttctccctgacatcc

SEQ ID NO: 18 - DNA, 22 bp, control konjugate
  Primer A2
attatagcctaagacccggagc

SEQ ID NO: 19 - DNA, 22 bp, artificial sequence
  Primer 3-1
ctggatctgaagctcccagatg

SEQ ID NO: 20 - DNA, 22 bp, artificial sequence
  Primer 3-2
tttcccctgatcagtctgggta

SEQ ID NO: 21 - DNA, 22 bp, artificial sequence
  Primer 3-3
gccatgggagaagaatatcccg

SEQ ID NO: 22 - DNA, 22 bp, artificial sequence
  Primer 3-4
ccatgcccacattcaggacttg

SEQ ID NO: 23 - DNA, 22 bp, artificial sequence
  Primer 3-5
ctaccaacttcacaccagcctg

SEQ ID NO: 24 - DNA, 22 bp, artificial sequence
  Primer 3-6
gggagagagaaaagaccatggg

SEQ ID NO: 25 - DNA, 22 bp, artificial sequence
  Primer 3-7N
tggaacaaggggtcagagtgat

SEQ ID NO: 26 - DNA, 23 bp, artificial sequence
  Primer 3-8
tggtctccactattcaagggaca

SEQ ID NO: 27 - DNA, 22 bp, artificial sequence
  Primer 3-9N
cacccccatatcccacatctaa

SEQ ID NO: 28 - DNA, 18 bp, artificial sequence
  Primer hyp0
tcagatgctctgtgcctt

SEQ ID NO: 29 - DNA, 18 bp, artificial sequence
  Primer hyb1
acacagtgtccaccggat

SEQ ID NO: 30 - DNA, 18 bp, artificial sequence
  Primer hyb2
cctgcaggtggatcgagt

SEQ ID NO: 31 - DNA, 18 bp, artificial sequence
  Primer hyb03
tgagttgcactgtgtaag

SEQ ID NO: 32 - DNA, 18 bp, artificial sequence
  Primer hyb4
ttaggtgagagcagtgtc

SEQ ID NO: 33 - DNA, 18 bp, artificial sequence
  Primer hyb5
catggccaagccagaaac

SEQ ID NO: 34 - DNA, 18 bp, artificial sequence
  Primer hyb6
tacacctctctgtcccca

SEQ ID NO: 35 - DNA, 18 bp, artificial sequence
  Primer hyb7
gcgtctacagggacacag